(19) United States
(12) Patent Application Publication
(10) Pub. No.: US 2026/0325175 A1
(43) Pub. Date: Oct. 22, 2026

(54) PNEUMATIC COMPRESSION DEVICE FOR POPLITEAL CYST

(71) Applicant: The Second Affiliated Hospital, Guangzhou Medical University, Guangzhou City (CN)

(72) Inventors: Yongzhen TAN, Guangzhou City (CN); Jingzhi ZHANG, Guangzhou City (CN); Bin LIU, Guangzhou City (CN); Bo DENG, Guangzhou City (CN); Huafeng LI, Guangzhou City (CN); Shuangwei ZHANG, Guangzhou City (CN); Jingyang ZHONG, Guangzhou City (CN)

(21) Appl. No.: 19/701,766

(22) Filed: Jun. 9, 2026

Related U.S. Application Data
(63) Continuation of application No. PCT/CN2024/122084, filed on Sep. 28, 2024.

(30) Foreign Application Priority Data
Aug. 27, 2024 (CN) 202411182693.0

(51) Int. Cl. A61B 17/56; A61B 17/00; A61B 17/12

(52) U.S. Cl. CPC A61B 17/562; A61B 2017/00544; A61B 2017/00557; A61B 2017/00876; A61B 2017/12018

(57) ABSTRACT

A pneumatic compression device for popliteal cyst includes an elastic band, two ends of a side of the elastic band are respectively detachably connected to two ends of a side of a fixing band via hook-and-loop fasteners, multiple inflatable bags are installed inside the fixing band, a magnetic sheet is installed on another side of the fixing band, first connecting tubes are fixedly connected to the side of the fixing band, an end of each of the first connecting tubes extending into the fixing band is in communication with a corresponding one of the inflatable bags, and another end of one of the first connecting tubes away from the inflatable bags is detachably connected to a control assembly.

### CROSS-REFERENCE TO RELATED APPLICATIONS

This application is a continuation of International Patent Application No. PCT/CN2024/122084, filed on Sep. 28, 2024, and claims priority of Chinese Patent Application No. 202411182693.0, filed on Aug. 27, 2024. The contents of International Patent Application No. PCT/CN2024/122084 and Chinese Patent Application No. 202411182693.0 are hereby incorporated by reference.

### TECHNICAL FIELD

The present disclosure relates to the technical field of popliteal cyst treatment, and in particular relates to a pneumatic compression device for popliteal cyst.

### BACKGROUND

A popliteal cyst refers to a general term for swelling of a deep bursa in a popliteal fossa or backward bulging of a synovial capsule of a knee joint, causing pain and distension in a posterior part of a knee, and a palpable elastic soft tissue mass. At present, a treatment method for a popliteal cyst is still dominated by popliteal cyst excision. A principle of non-surgical treatment of a popliteal cyst is: destroying a cyst wall, squeezing out cyst fluid, and applying pressure dressings. Non-surgical treatment includes manual therapy, acupuncture therapy, and block therapy. After a cyst wall is ruptured by the above three methods, appropriate pressure needs to be maintained at an affected part for about 4 weeks, so that cyst walls come into close contact, form adhesion, and avoid recurrence. Therefore, a pneumatic compression device for popliteal cyst is urgently needed to provide continuous sufficient pressure on a cyst surface, so that the cyst walls may stably fuse together, thereby achieving a curative effect.

### SUMMARY

An object of the present disclosure is to provide a pneumatic compression device for popliteal cyst, so as to solve the problems existing in the above prior art.

To achieve the above object, the present disclosure provides the following solution: a pneumatic compression device for popliteal cyst, including an elastic band, where two ends of a side of the elastic band are respectively detachably connected to two ends of a side of a fixing band via hook-and-loop fasteners, multiple inflatable bags are installed inside the fixing band, a magnetic sheet is installed on another side of the fixing band, first connecting tubes are fixedly connected to the side of the fixing band, an end of each of the first connecting tubes extending into the fixing band is in communication with a corresponding one of the inflatable bags, and another end of one of the first connecting tubes is detachably connected to a control assembly.

In an embodiment, a limiting hole is formed at a position, opposite to the magnetic sheet, of the elastic band, and the limiting hole is adapted to a knee.

In an embodiment, multiple accommodating cavities are formed in the fixing band, and the accommodating cavities are in one-to-one correspondence with the inflatable bags.

In an embodiment, the multiple accommodating cavities are evenly arranged inside the fixing band at equal intervals.

In an embodiment, a side of the magnetic sheet away from the fixing band is configured to contact a popliteal fossa.

In an embodiment, the control assembly includes a second connecting tube, the second connecting tube is in threaded connection with the one of the first connecting tubes, an end of the second connecting tube away from the one of the first connecting tubes is fixedly connected to and in communication with a hand-held inflatable bulb, and a pressure gauge is fixedly connected to and in communication with a portion of the second connecting tube adjacent to the hand-held inflatable bulb.

In an embodiment, a limiting groove is provided on an inner wall of each of the first connecting tubes, a sealing plug is slidably connected to the inner wall of each of the first connecting tubes, a movable disc is slidably connected to the limiting groove, an end of the movable disc facing the corresponding inflatable bag is fixedly connected to a spring, an end of the spring away from the movable disc is fixedly connected to the limiting groove, the sealing plug is fixedly connected to another end of the movable disc away from the spring.

In an embodiment, multiple vent grooves are formed in the movable disc, and the vent grooves are arranged around the sealing plug.

In an embodiment, the multiple vent grooves are provided at equal intervals.

In an embodiment, a fixing post is arranged inside an end of the second connecting tube facing the one of the first connecting tubes, the fixing post is fixedly connected to an inner wall of the second connecting tube via multiple connecting rods, and an end of the fixing post extending out of the second connecting tube is detachably connected to the sealing plug.

The present disclosure discloses the following technical effects:

The present disclosure installs the fixing band onto an affected area of a patient via the elastic band. At the same time, the present disclosure may control a size of the inflatable bag corresponding to the affected area via the control assembly. Thus, the inflatable bags may push the magnetic sheet to tightly abut against the affected area. The magnetic sheet may provide an appropriate continuous pressure to a cyst site. The magnetic sheet enables cyst walls of a ruptured cyst from which cyst fluid has been drained to adhere to each other, with no more cyst fluid exudation. At the same time, the magnetic sheet also provides a continuous physical therapeutic effect to the popliteal cyst, thereby achieving a curative effect, and effectively improving a cure rate.

### BRIEF DESCRIPTION OF THE DRAWINGS

In order to more clearly illustrate the embodiments of the present disclosure or the technical schemes in the prior art, a brief introduction will be given below to the drawings required for use in the embodiments. Apparently, the drawings in the following description are only some embodiments of the present disclosure, and for those of ordinary skill in the art, other drawings may also be obtained according to these drawings without expending creative labor.

FIG. 1 is a schematic diagram of an overall structure of the present disclosure.

FIG. 2 is a schematic structural diagram of a fixing band of the present disclosure.

FIG. 3 is a schematic structural diagram of a rear view of the fixing band of the present disclosure.

FIG. 4 is a top sectional view of the fixing band of the present disclosure.

FIG. 5 is a schematic structural diagram of an inflatable bag of the present disclosure.

FIG. 6 is a sectional view of a first connecting tube of the present disclosure.

FIG. 7 is a schematic structural diagram of a movable disc of the present disclosure.

FIG. 8 is a sectional view of a second connecting tube of the present disclosure.

### DETAILED DESCRIPTION OF THE EMBODIMENTS

The technical schemes in the embodiments of the present disclosure will be clearly and completely described below in conjunction with the drawings in the embodiments of the present disclosure. Apparently, the described embodiments are only a part of the embodiments of the present disclosure, rather than all of the embodiments. All other embodiments obtained by those of ordinary skill in the art based on the embodiments of the present disclosure without expending creative labor fall within the protection scope of the present disclosure.

To make the above objects, features and advantages of the present disclosure more apparent and easier to understand, the present disclosure is further described in detail below in conjunction with the drawings and specific implementations.

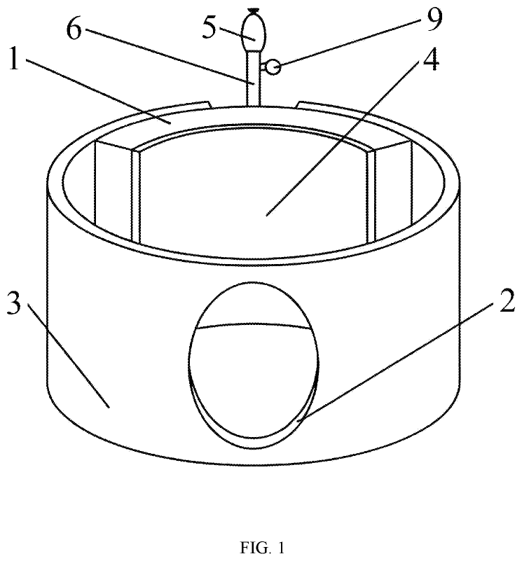

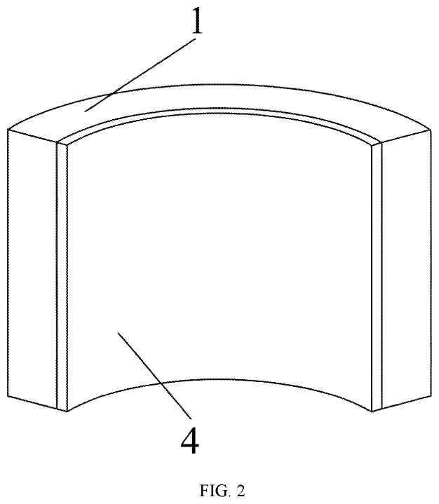

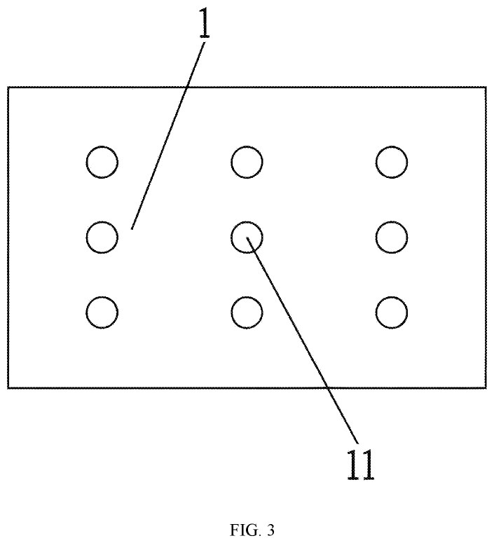

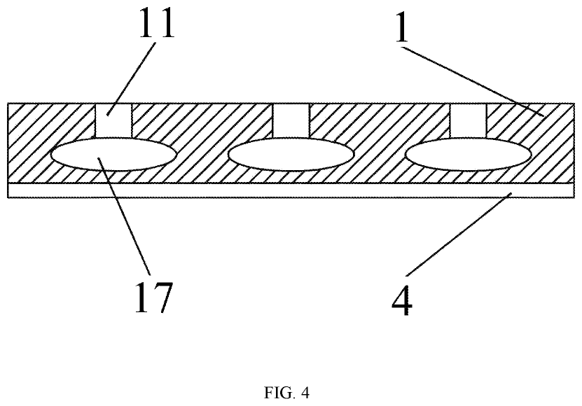

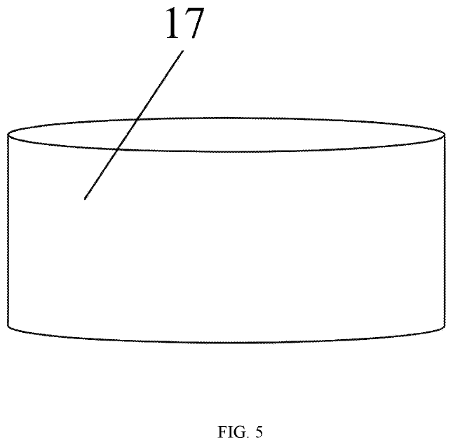

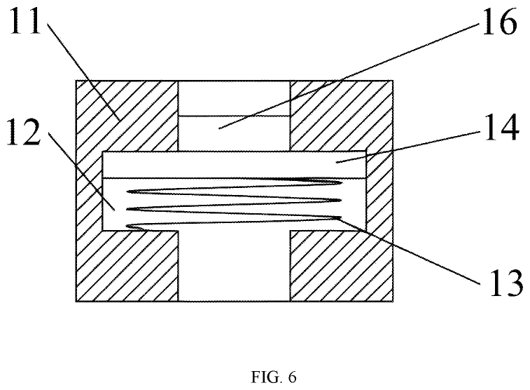

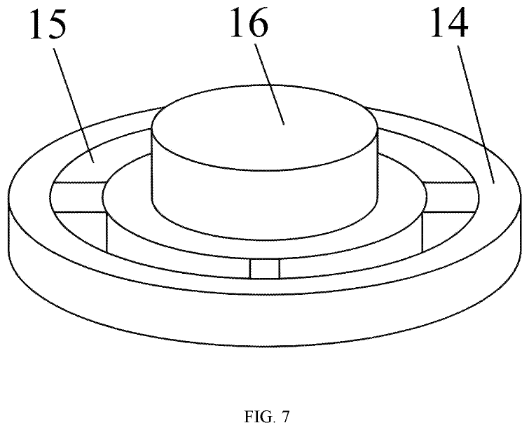

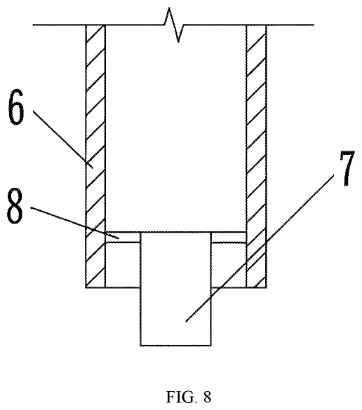

With reference to FIG. 1, FIG. 2, FIG. 3, FIG. 4, FIG. 5, FIG. 6, FIG. 7, and FIG. 8, the present disclosure provides a pneumatic compression device for popliteal cyst, including an elastic band 3. Two ends of a side of the elastic band 3 are respectively detachably connected to two ends of a side of a fixing band 1 via hook-and-loop fasteners, accommodating cavities are provided in the fixing band 1, inflatable bags 17 are respectively arranged in the accommodating cavities, a magnetic sheet 4 is installed on the other side of the fixing band 1, the magnetic sheet 4 is detachably connected to the fixing band 1, first connecting tubes 11 are fixedly connected to the side of the fixing band 1 away from the magnetic sheet 4. An end of the first connecting tube 11 extending into the accommodating cavity is in communication with the inflatable bag 17, and the other end of the first connecting tube 11 away from the inflatable bag 17 is detachably connected to a control assembly.

In the embodiment of the present disclosure, the fixing band 1 is installed onto an affected area of a patient via the elastic band 3, and a size of the inflatable bag 17 corresponding to the affected area is controlled via the control assembly. Thus, the inflatable bag 17 may push the magnetic sheet 4 to tightly abut against the affected area. The magnetic sheet 4 may provide an appropriate continuous pressure to a cyst site. The magnetic sheet 4 enables cyst walls of a ruptured cyst from which cyst fluid has been drained to adhere to each other, with no more cyst fluid exudation. At the same time, the inflatable bag 17 pushes the magnetic sheet 4. Thus, the magnetic sheet 4 may provide continuous sufficient pressure on a cyst surface. Cyst walls of the cyst are promoted to adhere to each other, and a curative effect is achieved. The magnetic sheet 4 closely attached to the skin also provides a continuous physical therapeutic effect to the popliteal cyst, thereby achieving the curative effect, and effectively improving a cure rate.

The fixing band 1 is an elastic bandage. The fixing band 1 provides fixation. The fixing band 1 may not only effectively fix the magnetic sheet 4 at the affected area, but can also protect blood vessels, nerves, muscles, skin and other soft tissues. The tightness may be adjusted through the hook-and-loop fastener, and adjustment may be carried out according to different limb thicknesses of different patients. The fixing band 1 and the elastic band 3 are installed and detached by means of the hook-and-loop fastener, and may adapt to knee joints with different circumferences and carry out effective fixation.

The inflatable bag 17 is made of a rubber material, which effectively avoids skin compression injury and allergy, and the inflatable bag 17 is in an elliptical flat shape.

In the embodiment, a limiting hole 2 is formed at a position, opposite to the magnetic sheet 4, of the elastic band 3, and the limiting hole 2 is adapted to a knee. When a knee of a patient is inside the limiting hole 2, the limiting hole 2 enables the knee of the patient to be positioned inside the limiting hole 2, so that the fixing band 1 may be stably located at the affected area of the patient, and the fixing band 1 would not disengage from the affected area.

In the embodiment, the accommodating cavities are in one-to-one correspondence with the inflatable bags 17.

In the embodiment, the accommodating cavities are evenly arranged inside the fixing band 1 at equal intervals. Due to different positions of affected areas of patients, the magnetic sheet 4 may be effectively and closely attached to an affected area through the accommodating cavities.

In the embodiment, the side of the magnetic sheet 4 away from the fixing band 1 may contact a popliteal fossa.

The inflatable bag 17 corresponding to the affected area is inflated, and the inflatable bag 17 expands and then squeezes the magnetic sheet 4, so that the magnetic sheet 4 may be closely attached to the affected area of a patient.

The magnetic sheet 4 is a cloth sheet with magnetic powder, and a size of the magnetic sheet 4 may be adjusted according to the affected area. The magnetic sheet 4 may cover the affected area, and may have a therapeutic effect on a local popliteal cyst.

The inflatable bag 17 pushes the magnetic sheet 4 to provide an appropriate pressure to the cyst site, so that cyst walls of the ruptured cyst from which cyst fluid has been drained are fully adhered to each other, and no more cyst fluid exudes, thereby achieving a curative result.

In the embodiment, the control assembly includes a second connecting tube 6, the second connecting tube 6 is in threaded connection with the first connecting tube 11, an end of the second connecting tube 6 away from the first connecting tube 11 is fixedly connected to and in communication with a hand-held inflatable bulb 5, and a pressure gauge 9 is fixedly connected to and in communication with a portion of the second connecting tube 6 near the hand-held inflatable bulb 5.

The first connecting tube 11 is in threaded connection with the second connecting tube 6, and after the inflation of the inflatable bag 17 is completed via the hand-held inflatable bulb 5, the second connecting tube 6 is unscrewed to prevent the hand-held inflatable bulb 5 from hindering movement of a patient, so that the second connecting tube 6 and the hand-held inflatable bulb 5 would not hinder the movement of the patient.

The inflation pressure of the inflatable bag 17 may be controlled through the pressure gauge 9.

In the embodiment, a limiting groove 12 is provided on an inner wall of the first connecting tube 11, a movable disc 14 is slidably connected to the limiting groove 12, an end of the movable disc 14 facing the inflatable bag 17 is fixedly connected to a spring 13, an end of the spring 13 away from the movable disc 14 is fixedly connected to the limiting groove 12, a sealing plug 16 is fixedly connected to an end of the movable disc 14 away from the spring 13, and the sealing plug 16 is slidably connected to the inner wall of the first connecting tube 11. An outer diameter of the sealing plug 16 is adapted to an inner diameter of the first connecting tube 11.

In the embodiment, vent grooves 15 are provided on the movable disc 14, and the vent grooves 15 are arranged around the sealing plug 16. Gas inside the second connecting tube 6 may enter the first connecting tube 11 through the vent grooves 15, or gas inside the first connecting tube 11 may enter the second connecting tube 6 through the vent grooves 15.

In the embodiment, the multiple vent grooves 15 are provided at equal intervals.

In the embodiment, a fixing post 7 is arranged inside the end, facing the first connecting tube 11, of the second connecting tube 6, the fixing post 7 is fixedly connected to an inner wall of the second connecting tube 6 via multiple connecting rods 8, and an end of the fixing post 7 extending out of the second connecting tube 6 is detachably connected to the sealing plug 16.

A length of the fixing post 7 extending out of the second connecting tube 6 is greater than a length of the sealing plug 16, so that the fixing post 7 may push the sealing plug 16 into the limiting groove 12.

To prevent gas inside the inflatable bag 17 from being discharged from the first connecting tube 11 after the second connecting tube 6 is removed, the elasticity of the spring 13 drives the movable disc 14 to move, so that the movable disc 14 may insert the sealing plug 16 into the first connecting tube 11 and abut against the inner wall of the first connecting tube 11, and discharge of gas inside the inflatable bag 17 through the first connecting tube 11 is effectively prevented.

When inflation is needed, the second connecting tube 6 is tightened onto the first connecting tube 11, and at this time, the fixing post 7 may push the sealing plug 16, so that the sealing plug 16 does not block the inner wall of the first connecting tube 11, and gas sequentially passes through the vent grooves 15 and the first connecting tube 11 to enter the inflatable bag 17.

When deflation of the inflatable bag 17 is needed, the second connecting tube 6 is tightened onto the first connecting tube 11, and at this time, the fixing post 7 may push the sealing plug 16, so that the sealing plug 16 does not block the inner wall of the first connecting tube 11, then a deflation valve of the hand-held inflatable bulb 5 is opened, and gas inside the inflatable bag 17 sequentially passes through the first connecting tube 11, the vent grooves 15, and the second connecting tube 6 and is discharged from the deflation valve of the hand-held inflatable bulb 5.

A working process is as follows. The fixing band 1 is first placed at the popliteal fossa of the patient, and the magnetic sheet 4 abuts against the popliteal fossa. At the same time, a knee is positioned inside the limiting hole 2, and tightness adjustment is carried out by installing two ends of the elastic band 3 onto positions on the fixing band 1. Then the inflatable bag 17 corresponding to the affected area is determined. The second connecting tube 6 is tightened onto the first connecting tube 11 corresponding to that inflatable bag 17. The hand-held inflatable bulb 5 is squeezed to inflate the inflatable bag 17. The pressure of the inflatable bag 17 may be checked through the pressure gauge 9. After the magnetic sheet 4 tightly abuts against the popliteal fossa through the inflatable bag 17, the second connecting tube 6 is unscrewed, and the patient may then perform activities. At the same time, the magnetic sheet 4 is squeezed by the inflatable bag 17, the magnetic sheet 4 may abut against the affected area, so as to continuously treat the affected area. Through the actions of the limiting hole 2 and the inflatable bag 17, the fixing band 1 may effectively remain at the popliteal fossa when the patient moves. The magnetic sheet 4 may effectively and closely fit with the affected area. A treatment effect on the affected area is thereby improved.

When the fixing band 1 needs to be removed, the second connecting tube 6 is tightened onto the first connecting tube 11, and the deflation valve of the hand-held inflatable bulb 5 is opened. Gas inside the inflatable bag 17 sequentially passes through the first connecting tube 11, the vent grooves 15, and the second connecting tube 6 and is discharged from the deflation valve of the hand-held inflatable bulb 5, and then the elastic band 3 is detached from the fixing band 1.

In the description of the present disclosure, what needs to be understood is that orientations or positional relationships indicated by the terms “longitudinal”, “transverse”, “upper”, “lower”, “front”, “rear”, “left”, “right”, “vertical”, “horizontal”, “top”, “bottom”, “inner”, “outer” and the like are based on orientations or positional relationships shown in the drawings, and are only for the convenience of describing the present disclosure, rather than indicating or implying that a referred device or element must have a specific orientation, be constructed and operated in a specific orientation, and therefore may not be construed as a limitation on the present disclosure.

The embodiments described above are only descriptions of optional modes of the present disclosure, and are not limitations on the scope of the present disclosure. Various deformations and improvements made to the technical schemes of the present disclosure by those of ordinary skill in the art without departing from the design spirit of the present disclosure shall all fall within the protection scope determined by the claims of the present disclosure.

What is claimed is:

1. A pneumatic compression device for popliteal cyst, comprising: an elastic band, wherein two ends of a side of the elastic band are respectively detachably connected to two ends of a side of a fixing band via hook-and-loop fasteners, a plurality of inflatable bags are installed inside the fixing band, a magnetic sheet is installed on another side of the fixing band, first connecting tubes are fixedly connected to the side of the fixing band, an end of each of the first connecting tubes extending into the fixing band is in communication with a corresponding one of the inflatable bags, and another end of one of the first connecting tubes is detachably connected to a control assembly.

2. The pneumatic compression device for popliteal cyst according to claim 1, wherein a limiting hole is formed at a position, opposite to the magnetic sheet, of the elastic band, and the limiting hole is adapted to a knee.

3. The pneumatic compression device for popliteal cyst according to claim 1, wherein a plurality of accommodating cavities are formed in the fixing band, and the accommodating cavities are in one-to-one correspondence with the inflatable bags.

4. The pneumatic compression device for popliteal cyst according to claim 3, wherein the accommodating cavities are evenly arranged in the fixing band at equal intervals.

5. The pneumatic compression device for popliteal cyst according to claim 1, wherein a side of the magnetic sheet away from the fixing band is configured to contact a popliteal fossa.

6. The pneumatic compression device for popliteal cyst according to claim 1, wherein the control assembly comprises a second connecting tube, the second connecting tube is in threaded connection with the one of the first connecting tubes, an end of the second connecting tube away from the one of the first connecting tubes is fixedly connected to and in communication with a hand-held inflatable bulb, and a pressure gauge is fixedly connected to and in communication with a portion of the second connecting tube adjacent to the hand-held inflatable bulb.

7. The pneumatic compression device for popliteal cyst according to claim 6, wherein a limiting groove is provided on an inner wall of each of the first connecting tubes, a sealing plug is slidably connected to the inner wall of each of the first connecting tubes, a movable disc is slidably connected to the limiting groove, an end of the movable disc facing the corresponding one of the inflatable bags is fixedly connected to a spring, an end of the spring away from the movable disc is fixedly connected to the limiting groove, the sealing plug is fixedly connected to another end of the movable disc away from the spring.

8. The pneumatic compression device for popliteal cyst according to claim 7, wherein a plurality of vent grooves are formed in the movable disc, and the vent grooves are arranged around the sealing plug.

9. The pneumatic compression device for popliteal cyst according to claim 8, wherein the vent grooves are arranged at equal intervals.

10. The pneumatic compression device for popliteal cyst according to claim 7, wherein a fixing post is arranged in an end, facing the one of the first connecting tubes, of the second connecting tube, the fixing post is fixedly connected to an inner wall of the second connecting tube via a plurality of connecting rods, and an end of the fixing post extending out of the second connecting tube is detachably connected to the sealing plug.
